(19) United States
(12) Patent Application Publication
(10) Pub. No.: US 2011/0295240 A1
(43) Pub. Date: Dec. 1, 2011

(54) FOLEY CATHETER WITH PROXMITY SENSOR

(75) Inventors: Kory P. Hamel, Bloomington, MN (US); Adam L. Gullickson, Richfield, MN (US)

(73) Assignee: AMS Research Corporation, Minnetonka, MN (US)

(21) Appl. No.: 13/141,417

(22) PCT Filed: Dec. 21, 2009
(86) PCT No.: PCT/US2009/069016

§ 371 Date: Aug. 22, 2011

Related U.S. Application Data
(60) Provisional application No. 61/140,151, filed on Dec. 23, 2008.

(51) Int. Cl. A61M 25/01

(52) U.S. Cl. 604/544

(57) ABSTRACT

An improved catheter design and related methods providing for a sensor disposed within a sensor lumen to provide detection and warning of instruments placed within the proximity of various anatomical structures. The sensor lumen can extend axially the length of a drainage lumen. The sensor or sensors should be able to provide detection warnings for the entire region. The sensor will activate a visual sensor, for example, an LED, an audible sensor, for example, a siren, or similar type alert notification systems. The detecting and indicating means can generate an indicator signal indicative of the presence and/or absence of the instrument in the lumen, and generally provides feedback to the surgeon.

### PRIORITY CLAIM

The present application claims priority to U.S. Provisional Application Ser. No. 61/140,151, filed Dec. 23, 2008 and entitled “FOLEY CATHETER WITH PROXIMITY SENSOR”, which is herein incorporated by reference in its entirety.

### FIELD OF THE INVENTION

The present invention relates generally to catheters and procedures related to catheter placement. More specifically, the present invention is directed to a catheter device including sensors for detecting the proximity of instruments relative to the urethra, bladder and rectal wall so as to reduce the risk of puncture and lessen the reliance on image guidance.

### BACKGROUND OF THE INVENTION

A catheter is a tubular medical device for insertion into canals, vessels, passageways or body cavities as an access means for introduction or removal of fluids or as a spacer to maintain the shape of the passage. One typical use of catheters is to permit urinary bladder drainage in certain patients who have had major surgery and/or trauma, or in any patient unable to urinate. In many instances, the widely-used Foley catheter is inserted in the urethra and up to the bladder where a retention balloon is inflated.

Catheters are also used for treatment of the prostate and bladder. The catheter promotes healing after surgery as it is important to drain the bladder by an indwelling urinary catheter. In addition, thousands of prostatectomies are performed each year involving the use of a catheter. For example, in a radical prostatectomy, the surgeon removes all or most of the patient's prostate. Because the urethra travels through the prostate immediately before reaching the bladder, the upper part of the urethra is removed in the surgery. The procedure leaves a severed urethral stump and a severed bladder neck.

To restore proper urinary functions, the bladder and the urethra must be reconnected by a laparoscopic technique known as an urethrovesical anastomosis. Anastomosis is the union or joinder of one hollow vessel or structure to another so that the interior of the vessels communicate with one another. Conventionally, a surgeon can execute delicate suturing operations with tiny, fine needles to reconnect these anatomical bodies. Deployment of sutures, however, with a needle, to connect the severed tissues, can be a difficult and often a technique-sensitive task. Many factors can make this task difficult, including a very small amount of tissue to work with (at the urethral stump and at the bladder neck), proximal urethras at the bladder, and a proximal nerve bundle and sphincter at the urethral stump. All of these add up to a complicated and delicate suturing procedure that, if not performed properly, can result in complications such as leakage, difficulty in healing or failure to heal, incontinence, or impotence. Specific problems include necrosis of the sutured tissues, stricture of the urethra thereby by impeding the flow of fluid through it, and a urethra-bladder connection that is not fluid-tight. In addition, methods of suturing the urethra to the bladder allow for accidental or inadvertent piercing of the nearby neurovascular bundle, which also can cause incontinence or impotence.

Another typical procedure affecting males is the correction of incontinence. Incontinence is a condition characterized by involuntary loss of urine, beyond the individual's control, that results in the loss or diminution of the ability to maintain the urethral sphincter closed as the bladder fills with urine. Male stress urinary incontinence (SUI) occurs when the patient is physically or emotionally stressed. One cause for this condition is damage to the urethral sphincter or loss of support of the urethral sphincter, such as can occur in males after prostatectomy or following radiation treatment, or that can occur due to pelvic accidents and aging-related deterioration of muscle and connective tissue supporting the urethra. Other causes of male incontinence include bladder instability, over-flowing incontinence and fistulas.

One type of procedure, found to be an especially successful treatment option for SUI in both men and women, is a sling procedure. There are a variety of different sling procedures. Descriptions of different sling procedures are disclosed in U.S. Pat. Nos. 5,112,344; 5,611,515; 5,842,478; 5,860,425; 5,899,909; 6,039,686, 6,042,534 and 6,110,101. A sling procedure is a surgical method involving the placement of a sling through the use of needle-like instruments to stabilize or support the bladder neck or urethra. In the male case, this sling applies passive compression against the bulbar urethra. The compression, either by itself or in conjunction with urethral mobility, prevents urine leak during strain. If additional passive pressure is required on the urethra after surgery is completed, collagen or other suitable bulky materials can be injected with a tiny needle through the perineum, causing more pressure created by the bulky material held on one side (the lower side) by the sling, and on the other side compressing the urethra. One minimally invasive surgical procedure to alleviate mild to moderate male SUI is performed employing the InVance™ Male Urethral Sling System for implanting the InteMesh™ Synthetic Surgical Mesh (both available from American Medical Systems, Inc.). As with the prostate treatment described above, a Foley catheter, for example, is inserted into the urethra, and the catheter balloon is inflated to assist the surgeon in identifying the urethra during the procedure.

Although serious complications associated with sling procedures are infrequent, they do occur. Complications associated with known sling procedures include injury to blood vessels of the pelvic sidewall and abdominal wall, hematomas, urinary retention, and bladder and bowel injury due to passage of large needles. Further, a separate cystoscopy procedure is usually required in order to confirm bladder integrity or recognize a bladder perforation after each insertion of the needle-like element.

Thus it is clear there is an unmet need for an improved apparatus and technique for monitoring the proper placement and location of the instruments used to correct incontinence and prostate disorders.

### SUMMARY OF THE INVENTION

The present invention is a sensorized Foley catheter. As minimally invasive procedures continue to develop for treatment of the urinary tract, prostate and erectile restoration, it is important to provide a safety system so as to not accidentally injure surrounding tissue. Specifically, in procedures to restore proper urinary functions, the bladder and the urethra must be reconnected by a laparoscopic technique known as an urethrovesical anastomosis. Anastomosis is the union or joinder of one hollow vessel or structure to another so that the interior of the vessels communicate with one another. Conventionally, a surgeon can execute delicate suturing operations with tiny, fine needles to reconnect these anatomical bodies. Installation of sutures, however, with a needle, to connect the severed tissues can be a difficult and often a technique-sensitive task. Likewise, the placement of a sling for treating incontinence requires the use of needles to position the sling material about the urethra. The Foley catheter is used in both procedures to drain the bladder and provide a reference point. As the Foley catheter is a common element to both procedures it is advantageous to have a detection component built into the catheter to notify the surgeon when an instrument is close to the urethra or rectum and to decrease the surgeon's reliance on image guidance for detection of the location of instruments.

In one aspect, the present invention involves a Foley catheter including one or more sensors positioned within a sensor lumen along the length of the catheter. The sensor lumen would extend axially the length of the drainage lumen. The sensor or sensors should be able to provide detection warnings for the entire region. The sensor will activate a visual sensor, for example, an LED, an audible sensor, for example, a siren, or similar type alert notification systems. The detecting and indicating means can utilize a conductive needle, a mechanical switch, a magnetic switch, a Hall effect sensor, an electric field, a magnetic field, or an inductor, for example, to detect the instruments. The detecting and indicating means can generate an indicator signal indicative of the presence and/or absence of the instrument in the lumen, and generally provides feedback to the surgeon.

In another aspect, the present invention is directed to a method for monitoring the presence and positioning of an instrument within a catheter. The method can comprise the step of providing a catheter having a sensor, instrument and drainage lumens extending the length of the catheter. The method can further comprise positioning one or more sensors within the sensor lumen. The method can further comprise detecting the present of an instrument within the instrument lumen using the one or more sensors.

The above summary of the invention is not intended to describe each illustrated embodiment or every implementation of the present invention. The Figures and the detailed description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of Foley catheter incorporating the present invention.

FIG. 2 is a cross sectional view of the catheter of FIG. 1 along section 2-2 of FIG. 1.

FIG. 3 is a perspective view of a section of the catheter of FIG. 1 with a sensor lumen in phantom.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

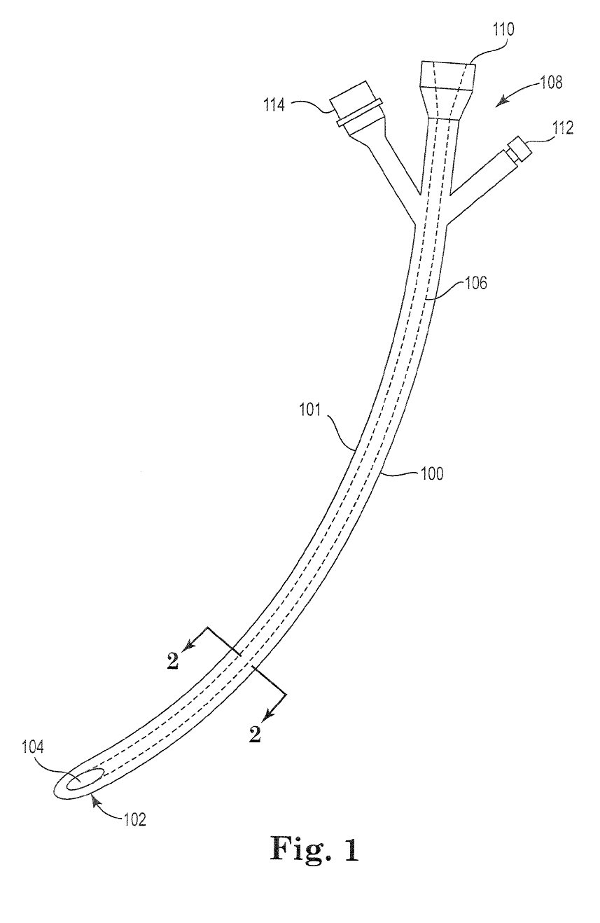

FIG. 1 is a perspective view of a Foley catheter 100 according to an embodiment of the present invention. Catheter 100 includes an elongate catheter body 101 having a distal end 102 and a proximal end 110. Distal end 102 can have an access port 104 fluidly connected to a drainage lumen 106 that extends through the catheter body 101 from the distal end 102 to the proximal end 108. The proximal end 108 can include a drainage port 110, a sensor port 112 and an instrument port 114. Drainage port 110 can be fluidly connected with drainage lumen 106 to allow drainage of fluids entering access port 104.

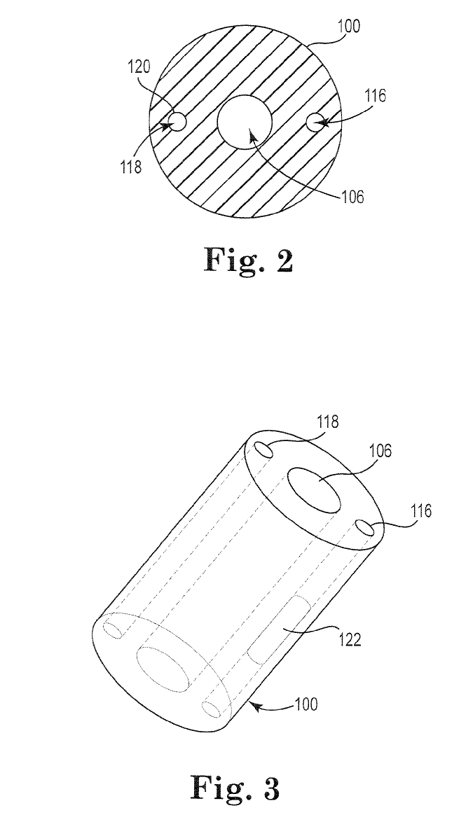

FIG. 2 is a cross sectional view of the catheter 100 of FIG. 1 at section 2-2. The catheter 100 can include a drainage lumen 106, a sensor lumen 116 and an instrument lumen 118. The instrument lumen 118 extends from the instrument port 114 to the distal end 102 at which an injector port 120 or similar opening may provide access into the body of a patient from instrument lumen 118. Instrument lumen 118 can be configured to allow passage of needles and other instruments used to conduct procedures with catheters 100. Other potential instruments include a fiber optic system extending the length of instrument lumen 118. The sensor lumen 116 connects to the sensor port 112. The sensor lumen 116 is depicted as a single lumen but it is envisioned that in other embodiments the sensor system could be a continuous coil or circuit so that multiple sensor lumens 116 extend the length of the catheter 100.

FIG. 3 is a perspective view of a section of the catheter 100. One or more sensors 122 can be inserted through the sensor port 112 and positioned within a sensor lumen 116 along the length of the catheter 100. The sensor lumen 116 can extend axially the length of the catheter 100 generally parallel to the drainage lumen 106 and instrument lumen 118. In one embodiment, the sensor 122 or sensors 122 extend the entire length of the catheter body 101 in order to provide detection warnings for the entire region of the catheter 100. In some embodiments, a single sensor 122 extends the entire length of catheter body 101. In other embodiments, multiple discrete sensors 122 extend the length of the elongate catheter body 101. With discrete sensors 122, the relative position of sensors 122 can be varied so as to allow a positioning reference point to be created, selected and adjusted. Regardless of the sensor configuration, one or more sensors 122 provides indication of where in a defined region of instrument lumen 118 that the instrument is positioned.

The sensor 122 can activate, for example, a visual sensor such as an LED, an audible sensor such as a siren, or other type of alert notification system. The alert notification system can be connected to the sensor port 112. The sensor can use various detecting means, including, for example, a conductive needle, a mechanical switch, a magnetic switch, a Hall Effect sensor, an electric field, a magnetic field, or an inductor, to detect instruments in the instrument lumen 118. The detecting and indicating means can generate an indicator signal indicative of the presence and/or absence of the instrument in a region of the lumen, and can provide feedback to the surgeon. In one embodiment, the sensor 122 can provide feedback as to the exact location of an instrument within the instrument lumen 118. In another embodiment, the sensor 122 only provides an alert in the event that an instrument is advanced beyond the perimeter (e.g., the distal end 102) of the catheter 100.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. A catheter system comprising:
an elongate catheter body having a proximal end and a distal end, the elongate catheter body having a sensor lumen extending there through from the proximal end to the distal end, an instrument lumen extending there through from the proximal end to the distal end and a drainage lumen extending there through from the proximal end to the distal end, wherein the sensor lumen, instrument lumen and drainage lumen are separated and individually defined between the proximate and distal ends of the catheter body, the elongate catheter body further including a sensor port at the proximal end in communication with the sensor lumen, an instrument port at the proximal end in communication with the instrument lumen and a drainage port at the proximal end in communication with the drainage lumen; and
at least one sensor disposed in the sensor lumen, the at least one sensor configured to detect the position and presence of an instrument disposed within a defined region of the instrument lumen.

2. The system of claim 1, wherein the at least one sensor extends generally the entire length of the sensor lumen.

3. The system of claim 1, wherein the sensor comprises a single sensing apparatus.

4. The system of claim 1, wherein the sensor comprises a plurality of sensing apparatuses, each sensing apparatus corresponding to different defined regions along the instrument lumen.

5. The system of claim 1, wherein the sensor includes an alert notification system.

6. The system of claim 5, wherein the alert notification system is selected from the group consisting of: a visual indicator system and an audio notification system.

7. The system of claim 1, wherein the sensor is configured to alert a user when the instrument extends beyond the distal end of the catheter body.

8. The system of claim 1, wherein the sensor senses the instrument using a detection means that utilizes at least one sensing apparatus selected from the group consisting of: a conductive needle, a mechanical switch, a magnetic switch, a Hall Effect sensor, an electric field, a magnetic field, and an inductor.

9. A catheter system comprising:
an elongate catheter body having a proximal end and a distal end, the elongate catheter body having a sensor lumen extending there through from the proximal end to the distal end, an instrument lumen extending there through from the proximal end to the distal end and a drainage lumen extending there through from the proximal end to the distal end, wherein the sensor lumen, instrument lumen and drainage lumen are separated and individually defined between the proximate and distal ends of the catheter body, the elongate catheter body further including a sensor port at the proximal end in communication with the sensor lumen, an instrument port at the proximal end in communication with the instrument lumen and a drainage port at the proximal end in communication with the drainage lumen; and
a means for detecting an instrument in defined regions within the instrument lumen, the means for detecting being disposed in the sensor lumen.

10. The system of claim 9, wherein the means for detecting includes an alert notification system.

11. The system of claim 10, wherein the alert notification system is selected from the group consisting of: a visual indicator system and an audio notification system.

12. The system of claim 9, wherein the means for detecting is configured to alert a user when the instrument extends beyond the distal end of the catheter body.

13. A method for monitoring the presence of an instrument within an instrument lumen of a Foley catheter, comprising:
providing a catheter having a sensor, instrument and drainage lumens extending the length of the catheter, wherein the sensor lumen, instrument lumen and drainage lumen are separated and individually defined between the proximate and distal ends of the catheter body;
positioning one or more sensors within the sensor lumen; and
detecting the present of an instrument within the instrument lumen using the one or more sensors.

14. The method of claim 13, further comprising:
inserting the catheter into a patient; and
receiving an indication of a defined region in the instrument lumen that the instrument is positioned.

15. The method of claim 14, wherein the step of receiving the indication includes receiving a visual indication of the defined region.

16. The method of claim 14, wherein the step of receiving the indication includes receiving an audio indication of the defined region.
